(19) United States
(12) Patent Application Publication
(10) Pub. No.: US 2003/0022921 A1
(43) Pub. Date: Jan. 30, 2003

(54) Stable pharmaceutical formulation comprising torsemide modification II

(76) Inventors: Minutza Leibovici, Netanya (IL); Ruth Tenengauzer, Hod HaSharon (IL); Mira Kopel, Pardess Hanna (IL); Judith Aronhime, Rehovot (IL); Marco Kordova, Kfar Saba (IL)

Correspondence Address: KENYON & KENYON, ONE BROADWAY, NEW YORK, NY, 10004, US

(21) Appl. No.: 10/071,423

(22) Filed: Feb. 8, 2002

Related U.S. Application Data
(63) Continuation-in-part of application No. 09/789,424, filed on Feb. 21, 2001.

(51) Int. Cl. A61K 31/44; C07D 213/70

(52) U.S. Cl. 514/346; 546/291

(57) ABSTRACT

Novel, stable pharmaceutical formulations for the oral administration of high purity torsemide modification II are disclosed. These formulations release high purity torsemide modification II in water at a constant and high purity rate, and the high purity torsemide modification II therein does not rearrange to torsemide modification I over time. Methods for their manufacture are also disclosed.

### CROSS-REFERENCE TO RELATED APPLICATION

This application is a continuation-in-part of the U.S. patent application Ser. No. 09/789,424, filed on Feb. 21, 2001, the content of which is incorporated herein by reference.

### FIELD OF THE INVENTION

The present invention generally relates to a pharmaceutical formulation of torsemide; more particularly to a stable pharmaceutical formulation comprising torsemide modification 1I.

### BACKGROUND OF THE INVENTION

1-Isopropyl-3-[(4-m-toluidino-3-pyridyl)-sulfonyl]urea, represented by the following structural formula

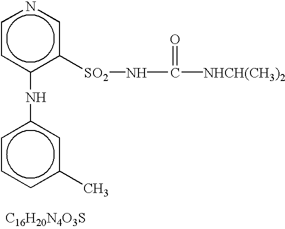

is approved under the trademark DEMADEX® by the U.S. Food and Drug Administration. DEMADEX® is clinically used in the treatment of hypertension and edema associated with congestive heart failure, renal disease, and hepatic disease. The USAN approved generic name for this compound is torsemide, although this compound is also referred to as “torasemide” in the art. Torsemide is a loop diuretic that has been found to be particularly effective for the treatment of edema associated with chronic renal failure.

U.S. Pat. No. Re. 30,633 describes the synthesis of torsemide. It is known that torsemide can occur in at least two different crystalline forms, Acta Cryst. 1978, pp. 2659-2662 and Acta Cryst., 1978, pp. 1304-1310, in which the crystal identified by space group P2,/c is designated Dupont Form 1 herein and the crystal identified by space group P2/n is designated Dupont Form 2 herein.

U.S. Pat. No. 4,822,807, which reissued as U.S. Pat. No. Re. 34,672, describes two crystalline forms of torsemide, designated modification I and modification II. Torsemide modification I is defined herein as the torsemide characterized by the x-ray powder diffraction pattern of FIG. 1, in the 37 C.F.R. §1.132 declaration by Dr. Fritz Topfmeier filed on Dec. 30, 1987, which is located in the file wrapper of U.S. Pat. No. 4,822,807 (the “Topfmeier Declaration”). Torsemide modification II is defined herein as the torsemide characterized by the x-ray powder diffraction pattern of FIG. 2, in the Topfmeier Declaration. U.S. Pat. Nos. Re. 30,633; 4,822,807; Re. 34,672; the 37 C.F.R. §1.132 declaration by Dr. Fritz Topfineier filed on Dec. 30, 1987, which is located in the file wrapper of U.S. Pat. No. 4,822,807; Acta Cryst. 1978, pp. 2659-2662; and Acta Cryst., 1978, pp. 1304-1310, are all incorporated herein by reference.

U.S. Pat. No. 4,822,807 further describes that when torsemide modification II is present in very finely divided form in pharmaceutical tablets, it rearranges into torsemide modification I, with the result that the rate of dissolution of the active material upon introducing the tablets into water can be significantly changed. The dissolution rate is an important characteristics of a pharmaceutical dosage form and, in order to dose reproducibly, must not differ from one tablet to the next.

There remains a need in the art for pharmaceutical formulations containing torsemide modification II, wherein the torsemide modification II does not rearrange into torsemide modification I and remains stable with regard to dissolution rate.

### SUMMARY OF THE INVENTION

An object of the present invention is to provide a stable pharmaceutical formulation comprising torsemide modification II, where upon storage under stress conditions, the torsemide modification II does not substantially rearrange into torsemide modification I or any other forms of torsemide and to provide a stable pharmaceutical formulation that is stable with regard to dissolution rate in solution and has a stable dissolution profile.

An additional object of the present invention is to provide a stable pharmaceutical formulation comprising an effective amount of torsemide modification II and pharmaceutically acceptable excipients wherein the excipients have a low moisture content.

An additional object of the present invention is to provide a high purity torsemide modification II which is substantially free of other forms of torsemide and processes for making the high purity torsemide modification II.

An additional object of the present invention is to provide a high purity torsemide modification II that does not substantially rearrange into a different form of torsemide over time upon storage in bulk under stress conditions.

The present invention provides a process for making high purity torsemide modification II comprising the steps of:

(a) adding torsemide modification I to a solvent mixture comprising acetonitrile and water;

(b) isolating torsemide modification I;

(c) suspending the torsemide modification I of step (b) in water to form a solution;

(d) adjusting the solution of step (c) to a pH of about 10±10.2;

(e) filtering the solution of step (d);

(f) adjusting the solution of step (e) to a pH of 6.25±0.2; and

(g) isolating high purity torsemide modification II.

The present invention also provides a process for making high purity torsemide modification II wherein the high purity torsemide modification II is purified from crude modification II by the novel combination of two purification steps known in the art wherein the novel process comprises the steps of (1) reslurrying crude torsemide modification II followed by (2) crystallization to yield high purity torsemide modification II by the methods of U.S. patent application Ser. No. 09/638,106, filed Aug. 11, 2000, the content of which is incorporated herein by reference.

### BRIEF DESCRIPTION OF THE DRAWINGS

FIG. 1 depicts an x-ray powder diffraction pattern of a high purity torsemide modification II tablet.

FIG. 2 depicts an x-ray powder diffraction pattern of bulk high purity torsemide modification II.

FIG. 3 depicts an x-ray powder diffraction pattern of a placebo tablet corresponding to a tablet containing 100 mg of high purity torsemide modification II.

### DETAILED DESCRIPTION OF THE INVENTION

High Purity Torsemide Modification II

The present invention provides high purity torsemide modification II wherein the high purity torsemide modification II has the surprising and useful advantage of being a stable polymorphic form of torsemide, that is, it does not substantially rearrange over time, thereby making high purity torsemide modification II. Preferably, the high purity torsemide modification II does not substantially rearrange over time into torsemide modification I (such as not more than 10% of torsemide modification II rearranges to torsemide modification I).

The present invention provides a manufacture process of stable pharmaceutical tablets of torsemide modification II. Preferably, the high purity torsemide modification II is in the form of fine crystal. The high purity torsemide modification II of the present invention may be in the form of fine crystals. The high purity torsemide modification II may be further characterized by having a particle size distribution such that 100% is below 200μ. Preferably, the particle size distribution is such that 100% is below 100μ. More preferably, the particle size distribution is such that 100% is below 50μ. Fine crystals of the high purity torsemide modification II of the present invention having the desired particle size distribution can be obtained by the use of conventional techniques known in the art, for example, using a ball mill, ultrasonic means, using a jet mill, or other suitable means as disclosed in Pharmaceutical Dosage Forms: Tablets, Vol. 2, 2^{nd} Ed., Lieberman et al. Ed., Marcel Dekker, Inc, New York, (1990) p.107-200, the contents of which is incorporated herein by reference.

It was surprisingly found that when torsemide modification II is crystallized as high purity torsemide modification II, with no trace amounts of torsemide modification I, the high purity torsemide modification II is stable during storage under stress conditions for at least 3 months. In contrast, torsemide modification II that contains trace amounts of torsemide modification I is not stable during storage under stress condition for at least 3 months. The torsemide modification II containing trace amounts of torsemide modification I rearranges into torsemide modification I over time during storage under stress conditions.

Trace amounts, as defined herein, are the amounts of one polymorphic form that are about 0.5 to about 2% weight percent of the amount of the other polymorphic form present, e.g., w/w % of torsemide modification I/torsemide modification II. Significant rearrangement or substantial rearrangement, as defined herein, is any rearrangement of more than about 15% of one polymorphic form into any other different polymorphic form or amorphous form. into different polymorphic forms of torsemide. Preferably, not more than 10% of the high purity torsemide modification II rearranges into different polymorphic forms of torsemide.

Significantly, it has been found that upon storage at 40° C. at a 75% relative humidity for 3 months, the polymorphic content of high purity torsemide modification II of the tablet formulations, or the bulk active ingredient, does not undergo any significant rearrangement into different polymorphic forms of torsemide. Preferably, not more than 10% of the high purity torsemide modification II rearranges into different polymorphic forms of torsemide following storage of the tablets or bulk active ingredient. More preferably, not more than 5% of the high purity torsemide modification II rearranges into different polymorphic forms. Even more preferably, not more than 2% of the high purity torsemide modification II rearranges into different polymorphic forms and most preferably, the high purity torsemide modification II is substantially pure polymorph torsemide modification II following storage.

Specifically, the high purity torsemide modification II of the present invention does not undergo a polymorphic rearrangement into torsemide modification I. The detection of torsemide modification I in bulk high purity torsemide modification II or tablets of high purity torsemide modification II may be accomplished by using x-ray powder diffraction techniques. No substantial polymorphic change of the high purity torsemide modification II of the present pharmaceutical formulations or present bulk active ingredient can be detected by x-ray powder diffraction techniques.

Without being bound by theory, it is believed that the level of purity presently achieved in the high purity torsemide modification II imparts this polymorph with unexpected and beneficial stability. It is feasible that the unstable torsemide modification II described in the relevant art contains trace amounts of torsemide modification I, the presence of which facilitates the rearrangement of torsemide modification II into torsemide modification I. It has been reported in the art that trace amounts of torsemide modification I facilitates the conversation of torsemide modification II into torsemide modification I when in an aqueous suspension. Additionally, in solid-state reactions, it is known in the art that increasing the surface area of a solid, i.e. producing finer or smaller particles of a solid, generally functions to increase the rate of the solid-state reaction. Thus, in solid-state reactions, the rate of the reaction (presently, the speed of polymorphic rearrangement) would be relatively slow when using large crystals, and the rate of the reaction would generally be expected to increase as smaller and smaller crystals are used. This provides another feasible explanation of the observed rearrangement of finely divided torsemide modification II into torsemide modification I that has been reported in the art.

Stable Pharmaceutical Formulations

The present invention also relates to novel and stable pharmaceutical formulations containing fine crystals of high purity torsemide modification II wherein the present stable pharmaceutical formulations have the surprising and useful advantage that the active material, torsemide modification II, does not substantially rearrange into torsemide modification I (such as not more than 5% of torsemide modification II rearranges to torsemide modification I), thereby making the stable pharmaceutical formulations of the present invention useful for the administration of torsemide modification II. The pharmaceutical formulations of the present invention are solid dosage forms for the oral administration of torsemide that are presented as a tablet.

Surprisingly, it was also found that the pharmaceutical formulation containing use of Excipients with a low water content stabilizes modification II.

The present invention also provides new stable pharmaceutical formulations comprising an effective amount of torsemide modification II and pharmaceutically acceptable excipients wherein the excipients have a low moisture content. Preferably, the stable pharmaceutical formulation comprises the excipients lactose anhydrous, crospovidone, povidone, microcrystalline cellulose, and magnesium stearate all of which have a low moisture content. The choice and use of excipients that are anhydrous, having a lower water content than excipients more frequently used in the art, or excipients having the lowest water content available in the art, provides the surprising and advantageous stabilization of the torsemide modification II present in the stable pharmaceutical formulations of the present invention.

The present stable pharmaceutical formulations provide the surprising and beneficial characteristic that the torsemide modification II does not substantially rearrange into another form of torsemide over time. The other forms of torsemide, which are prevented from forming, are any torsemide molecule not having the polymorphic form of torsemide modification II, including, but not limited to, torsemide modification I, torsemide Form III, other polymorphic forms of torsemide reported in the art and amorphous torsemide.

Additionally, the present stable pharmaceutical formulations retain the beneficial characteristic of stabilizing torsemide modification II in the formulations by inhibiting the substantial rearrangement of torsemide modification II into another form of torsemide over time, even when stored under stress conditions for up to three months, e.g., 40° C., 75% relative humidity.

In another embodiment, the present invention provides the unexpected benefit of stabilizing finely divided torsemide modification II and thereby providing for stable pharmaceutical formulations of torsemide modification II wherein the torsemide modification II is present as fine crystals. Additionally, the present invention provides stable pharmaceutical formulations wherein the torsemide modification II has a particle size distribution such that 100% is below 200μ. Preferably, the particle size distribution is such that 100% is below 100μ. More preferably, the particle size distribution is such that 100% is below 50μ.

In another embodiment, the present invention provides stable pharmaceutical formulations of torsemide modification II having stable dissolution profiles. The present stable pharmaceutical formulations of torsemide modification II provide a dissolution rate in vitro, when measured by the U.S.P. Paddle Method at 50-90 RPM in 900 mL water, that is not less than 80% (by weight) of the torsemide modification II released after 30 minutes. Additionally, the present invention provides the unexpected and advantageous results for providing a stable pharmaceutical formulation of torsemide modification II having a dissolution rate in vitro that does not substantially change over time upon storage in bulk under stress conditions, e.g., 40° C., 75% relative humidity. Even more preferable, the present stable pharmaceutical formulation of torsemide modification II has a dissolution rate in vitro that does not substantially change during storage under stress conditions for at least 3 months

The present invention also provides methods for making stable pharmaceutical formulations of torsemide modification II, including torsemide modification II containing trace amounts of torsemide modification I, which are tablets. The present torsemide modification II tablets are prepared by mixing the active ingredient, torsemide modification II, with a combination of excipients including, lactose anhydrous NF, crospovidone NF, povidone USP (PVP K-30), and microcrystalline cellulose NF (Avicel PH 112). Alcohol 95% USP is added to the powder mixture of torsemide modification II and excipients. The mixture is then dried until only trace amounts of fluid remain in the granulate as residual moisture. Preferably, the mixture is dried to 0.5-1.5% moisture content. The granulate is then sieved, and magnesium stearate is added to the milled granulate. The final blend of torsemide modification II, excipients and magnesium stearate is compressed into tablets on a rotary tableting machine. Table 1 shows suitable ranges of active ingredients and excipients (weight %) and the preferred amounts for the present stable pharmaceutical formulations.

While not being bound by theory, it is believed that the observed unexpected stability of torsemide modification II (which is not high purity torsemide modification II) in the present pharmaceutical formulation is achieved by the present novel formulation which serves to inhibit the rearrangement of torsemide modification II into torsemide modification I.

TABLE 1

| | Range of % | Preferred % | |
|---|---|---|---|
| | composition | composition | |
| Material | (w/w) | (w/w) | Function |
| Torsemide modification II or | 2.5-25% | 5% | active |
| torsemide modification II with | | | ingredient |
| trace amounts of modifica- | | | |
| tion I | | | |
| Lactose Anhydrous NF | 25.5-65% | 45.5% | filler |
| Crospovidone NF | 10-15% | 12.0% | disintegrant |
| Povidone USP (PVP K-30) | 1-3% | 1.5% | binder |
| Microcrystalline Cellulose NF | 25-45% | 35.0% | filler and |
| (Avicel PH 112) | | | disintegrant |
| Alcohol 95% USP* | — | — | Granulation |
| | | | processing |
| | | | solvent |
| Magnesium Stearate NF | 0.5-2.5% | 1.0% | lubricant |

*Granulation processing solvent only (dried to achieve moisture content of 0.5-1.5%).

The present invention also provides methods for making stable pharmaceutical formulations of high purity torsemide modification II which are tablets. High purity torsemide modification II tablets are prepared by mixing the active ingredient, high purity torsemide modification II, with a combination of excipients including, lactose anhydrous NF, crospovidone NF, povidone USP (PVP K-30), and microcrystalline cellulose NF (Avicel PH 112). Alcohol 95% USP is added to the powder mixture of high purity torsemide modification II and excipients. The mixture is then dried until only trace amounts of fluid remain in the granulate as residual moisture. Preferably, the mixture is dried to about 0.5 to about 1.5% moisture content. The granulate is then sieved, and magnesium stearate is added to the milled granulate. The final blend of high purity torsemide modification II, excipients and magnesium stearate is compressed into tablets on a rotary tableting machine.

Table 2 shows suitable ranges of active ingredients and excipients (weight %) and the preferred amounts for the present pharmaceutical formulations.

TABLE 2

| | Range of % | Preferred % | |
|---|---|---|---|
| | composition | composition | |
| Material | (w/w) | (w/w) | Function |
| High purity Torsemide modi- | 2.5-25% | 5% | active |
| fication II | | | ingredient |
| Lactose Anhydrous NF | 25.5-65% | 45.5% | filler |
| Crospovidone NF | 10-15% | 12.0% | disintegrant |
| Povidone USP (PVP K-30) | 1-3% | 1.5% | binder |
| Microcrystalline Cellulose NF | 25-45% | 35.0% | filler and |
| (Avicel PH 112) | | | disintegrant |
| Alcohol 95% USP* | — | — | Granulation |
| | | | processing |
| | | | solvent |
| Magnesium Stearate NF | 0.5-2.5% | 1.0% | lubricant |

*Granulation processing solvent only (dried to achieve moisture content of 0.5-1.5%).

Surprisingly and significantly, it has also been found that the stable pharmaceutical formulations of the present invention containing fine crystals of high purity torsemide modification II have a dissolution rate in water and in potassium phosphate buffer that does not substantially change over time. It has been found that the tablet formulations of the present invention, during storage at 40° C., 75% relative humidity, for 6 weeks, do not undergo any substantial change in the dissolution rate. The dissolution rate was determined by the U.S.P. Paddle Method, 37° C., 90 RPM, 0.01M KH₂PO₄, pH 4.5; and by the U.S.P. Paddle Method, 37° C., 50 RPM, purified water.

Torsemide modification II suitable for use in the present stable pharmaceutical formulations includes high purity torsemide modification II; torsemide modification II containing trace amounts of torsemide modification I; fine crystals of high purity torsemide modification II; and fine crystals of torsemide modification II containing trace amounts of torsemide modification I. As specified above, trace amounts, as defined herein, are amounts of torsemide modification I that are about 0.5 to about 2% by weight of the torsemide modification II (w/w % of torsemide modification I/torsemide modification II).

The present invention also provides a process for making high purity torsemide modification II wherein the high purity torsemide modification II is purified from crude modification II by the novel combination of two purification steps known in the art wherein the novel process comprises the steps of (1) reslurrying crude torsemide modification II followed by (2) crystallization to yield high purity torsemide modification II by the methods of U.S. Ser. No. 09/638,106; filed Aug. 11, 2000, the contents of which are incorporated herein by reference. Crude torsemide modification II may be made by methods know in the art, such as disclosed in U.S. patent application Ser. Re. 30,633.

By the methods of the present invention, the high purity torsemide modification II, the torsemide modification I is prepared from crude torsemide modification II where the torsemide modification II is crude torsemide modification II; or mixtures of torsemide modifications I and II. The crude torsemide modification II is reslurried using a solvent mixture of acetonitrile and water, and the reaction is preferably stirred for greater than about 45 minutes.

In an embodiment of the present invention, the solvent mixture containing acetonitrile is: acetonitrile and water where the volume ratio is between about 1:15 and about 15:1. Preferably the acetonitrile to water ratio is about 5:1. Preferably, the reaction is stirred at room temperature until the reaction is complete. The completion of the reaction can be monitored by IR spectrometry. Torsemide modification I is isolated upon filtration and drying. The filtration may be done at a range of temperatures including from about 0° C. to about room temperature.

In the second step of the present method, the isolated torsemide modification I is then crystallized to yield the present high purity torsemide modification II. By the methods of the present invention the desired final product, high purity torsemide modification II, is isolated by adding the isolated torsemide modification I to water. The pH of the solution is then adjusted to about 10.2±0.2 with about 20% aqueous sodium hydroxide. The solution is then filtered and the pH of the solution was adjusted with approximately 66 mL of a 1:1 acetic acid:water solution to a pH of about 6.25±0.2. The white precipitate was filtered and washed with water (2×50 mL) and dried in a high vacuum oven at about 50° C. for about 6 hours. Torsemide modification II was isolated in 93.2% yield, 165 grams.

In accordance with the present invention, the pharmaceutical formulations of the present invention are useful for the treatment of hypertension and edema associated with congestive heart failure, renal disease, or hepatic disease. While one of ordinary skill in the art will understand that dosages will vary according to the indication, age of the patient, and other factors, generally the formulations of the present invention will be administered at a daily dosage of the active ingredient between about 2 to about 200 mg per day, and preferably about 5 mg to about 100 mg per day. As torsemide is suitable for once-daily dosing, preferably each unit dosage form will contain between about 5 mg and about 100 mg.

Additionally, the present invention provides stable pharmaceutical formulation comprises torsemide modification II in an amount of about 2.5 mg to about 200 mg per tablet. Preferably, the present invention provides stable pharmaceutical formulations comprising torsemide modification II in an amount of about 2.5 mg, about 5 mg, about 10 mg, about 20 mg or about 100 mg per tablet.

### EXAMPLES

The pursuant invention will now be further explained in the following examples. However, the present invention should not be construed as limited thereby.

TABLE 3
Determination of Polymorphic Content by XRPD in Bulk High Purity
Torsemide Modification II (Bulk Lot No. 851700100)

| | |
|---|---|
| Polymorph Content of Bulk | |
| High Purity Torsemide modi- | |
| fication II Storage Conditions | Length of |

| | | |
|---|---|---|
| 55° C. | 40° C., 75% RH | Storage |

| | |
|---|---|
| Polymorphic form detected (I or II)^{†} | t = 0 |

| | | |
|---|---|---|
| II | | 1 week |
| II | II | 2 weeks |
| II | II | 1 month |
| II | II | 2 months |
| II | II | 3 months |
| II | II | 4 months |

^{†}“I” is Torsemide Modification I; and “II” is Torsemide Modification II

TABLE 4
Determination of Polymorphic Content by XRPD Analysis Bulk
Torsemide Modification II (II) w/trace amounts of
Torsemide Modification I (I) (Bulk No. 851700200)

| | |
|---|---|
| Polymorph Content | Length of |
| Storage Conditions | Storage |
| II >>> I (<0.3%)^{†} | t = 0 |

| | | |
|---|---|---|
| II >> I (≈0.4%) | | 1 week |
| I + II | II >> I (≈0.5%) | 2 weeks |
| I + II | II >> I (≈1%) | 1 month |
| I + II | II >> I (≈6%) | 2 months |
| I > II | I + II | 3 months |
| I + II | I + II | 4 months |

^{†}“I” is Torsemide Modification I; and “II” is Torsemide Modification II

### Example 1

### X-Ray Powder Diffraction (XRPD) Method for the Detection and Quantification of Torsemide Modification I in Torsemide Modification II

1. The present procedure is used for the detection and quantitative determination of the presence of torsemide modification I in tablets wherein the active ingredient is high purity torsemide modification II. The present procedure is also used for the detection and quantitative determination of torsemide modification I in bulk high purity torsemide modification II, which is to be used as the active ingredient in tablets. The present method is based on the unique x-ray powder diffraction pattern of torsemide modification I that is characterized by a strong peak at two-theta 5.7±0.2°, the presence of which indicates the presence of torsemide modification I in a sample of high purity torsemide modification II.

2. EQUIPMENT

2.1 Instrument: Philips x-ray powder diffractometer. Goniometer model PW 1050/70, Cu-tube, curved graphite monochromator.

2.2 Sample holder: A standard aluminum sample holder with a rectangular cavity 20*15*0.3 mm inside it.

3. RUN PARAMETERS

Scanning range: 2θ=4° to at least 22°

Step: 0.05°

Step duration: 0.5

4. PROCEDURE FOR SAMPLE PREPARATION

4.1 Gently grind a small amount of sample powder in an agate mortar with the pestle.

4.2 Fill the rectangular cavity on the sample holder with the powder.

### Stability Results for Torsemide Tablets K-26058 and K-26683 Containing 100 mg of Active Ingredient

Tablets containing 100 mg of high purity torsemide modification II, prepared according to the methods of Example 2, were stored under stressed conditions (40° C., 75% relative humidity). The polymorphic content of torsemide inside the tablet was monitored by x-ray powder diffraction (XRPD) techniques. Representative x-ray powder diffraction patterns are shown in the Figures.

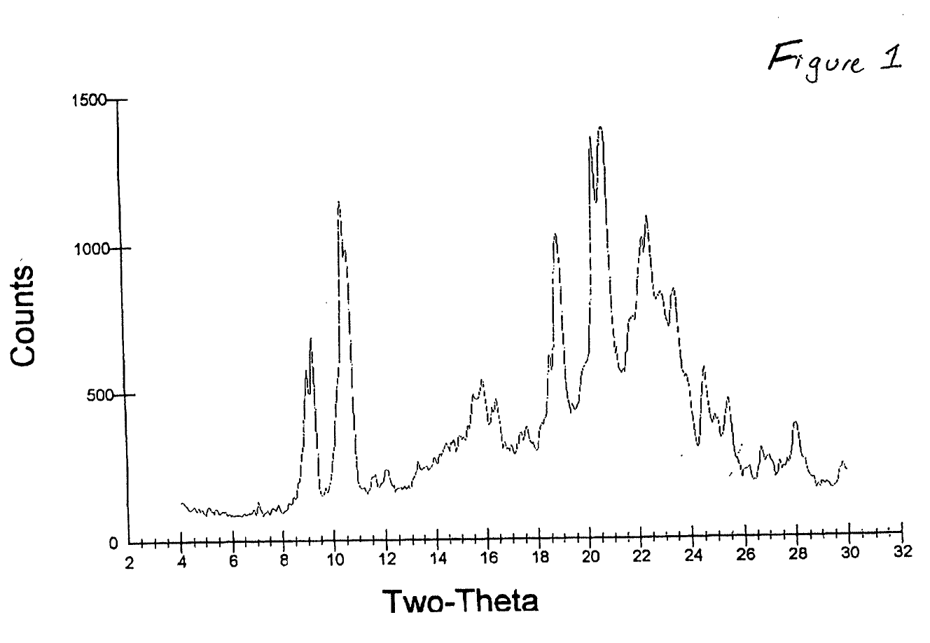

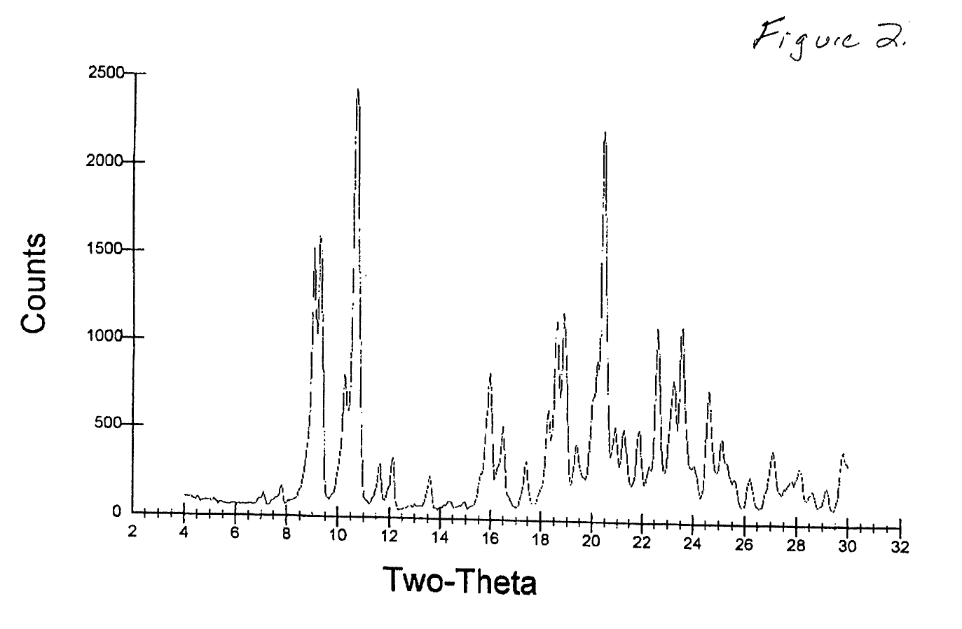

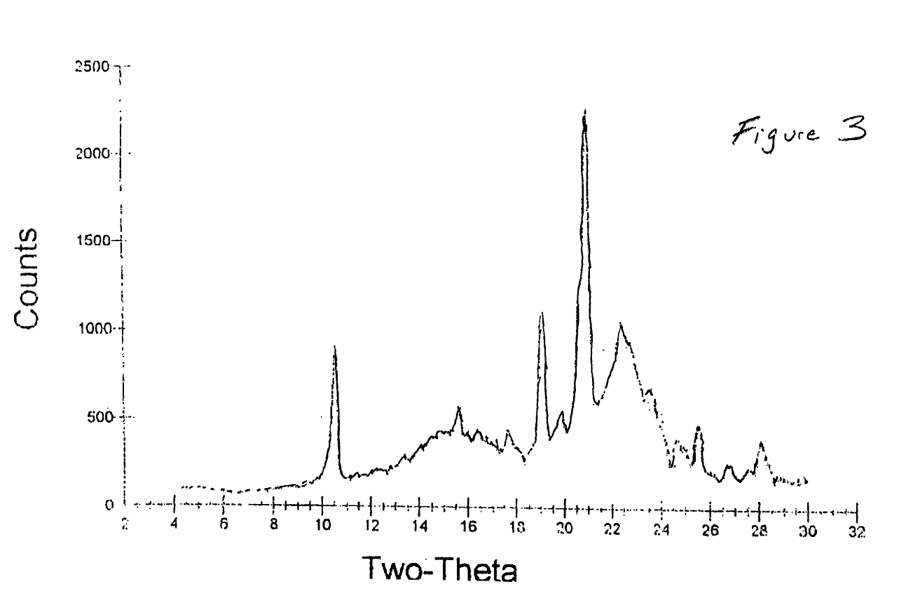

FIG. 1 is an x-ray powder diffraction pattern of a high purity torsemide tablet (Batch No. K-26683). FIG. 2 is an x-ray powder diffraction pattern of bulk high purity torsemide modification II (API 851700100). FIG. 3 is an x-ray powder diffraction pattern of a placebo tablet corresponding to a tablet containing 100 mg of high purity torsemide modification II and therefore contains no torsemide.

The XRPD of a 100 mg tablet of Batch No. K-26683 directly following production, t=0, showed XRPD peaks typical of high purity torsemide modification II. The XRPD of the K-26683 tablet following three months of storage at 40° C. and 75% relative humidity showed XRPD peaks typical of high purity torsemide modification II and did not show an XRDP peak at 5.7 degrees two-theta, which would indicate the presence of torsemide modification I. Similarly, the XRPD of a 100 mg tablet of Batch No. K-26058 directly following production, t=0, showed XRPD peaks typical of high purity torsemide modification II. The XRPD of the K-26058 tablet following three months of storage at 40° C. and 75% relative humidity showed XRPD peaks typical of torsemide modification II and did not show an XRDP peak at 5.7 degrees two-theta, which would indicate the presence of torsemide modification I. The diffraction peaks at 20.4 and the broad peak at about 22.5 degrees two-theta are characteristic of the filler.

Lower dosage tablets, for example, tablets containing 10 mg of high purity torsemide modification II, were stored for 2 months at 40° C., 75% relative humidity, and were monitored by solid state NMR. The resulting solid state NMR data indicated that the high purity torsemide modification II of the lower dose tablets did not substantially rearrange.

### Example 2

### Manufacturing Procedure

In a high speed mixer, high purity torsemide modification II was mixed with lactose anhydrous NF, crospovidone NF, povidone USP, and microcrystalline cellulose NF. Alcohol 95% USP was added to the powder mixture. The wet granulate mixture was dried in a fluid bed drier at 50° C. to a loss on drying (LOD) of 0.5-2.0%. The resulting dry granulate of high purity torsemide modification II was then sifted through a 0.8 mm sieve and magnesium stearate NF was added to the milled granulate. The final blend of high purity torsemide modification II, excipients and magnesium stearate was compressed into oval shaped tablets on a rotary tableting machine.

EXAMPLE 2A
High purity Torsemide Tablets (2.5 mg)

| Composition (Batch No. K-26056) | grams per 15,000 tablets |
|---|---|
| High Purity Torsemide Modification II | 37.5 |
| Lactose Anhydrous NF | 697.5 |
| Crospovidone NF | 150.0 |
| Povidone USP (PVP K-30) | 37.5 |
| Microcrystalline Cellulose NF (Avicel PH 112) | 52.5 |
| Alcohol USP | 500.0 |
| Magnesium Stearate NF | 12.8 |

EXAMPLE 2B
High purity Torsemide Tablets (5 mg)

| Composition (Batch No. K-26057) | grams per 15,000 tablets |
|---|---|
| High Purity Torsemide Modification II | 75 |
| Lactose Anhydrous NF | 697.5 |
| Crospovidone NF | 150.0 |
| Povidone USP (PVP K-30) | 37.5 |
| Microcrystalline Cellulose NF (Avicel PH 112) | 52.5 |
| Alcohol USP | 510.0 |
| Magnesium Stearate NF | 14.6 |

EXAMPLE 2C
High purity Torsemide Tablets (100 mg)

| Composition (Batch No. K-26058) | grams per 3,750 tablets |
|---|---|
| High Purity Torsemide Modification II | 375.0 |
| Lactose Anhydrous NF | 547.0 |
| Crospovidone NF | 150.0 |
| Povidone USP (PVP K-30) | 37.5 |
| Microcrystalline Cellulose NF (Avicel PH 112) | 375 |
| Alcohol USP | 616.0 |
| Magnesium Stearate NF | 15.0 |

### Example 3

### Dissolution Results

The dissolution method used was the U.S.P. Paddle Method, at 90 RPM with 0.1 M KH₂PO₄, pH 4.5 at 37° C. For the dissolution test, 6 tablets were tested in 900 mL of phosphate buffer, pH 4.5, according to the Paddle Method of the U.S.P. Examples 3A, 3B and 3C show the dissolution rates of three tablet lots directly after production and after 6 weeks of storage at 40° C. at a relative humidity (RH) of 75%. The dissolution rates of high purity torsemide Form II Batch Nos. K-26056, K-26057 and K-26058 were identical under both conditions. There was no substantial change in the dissolution rates of any of the present pharmaceutical formulations containing torsemide modification II following 6 weeks of the above storage conditions.

EXAMPLE 3A
Dissolution of 2.5 mg High Purity Torsemide modification II Tablets

| | Torsemide dissolved (%) | |
|---|---|---|
| Time | K-26056 (2.5 mg) | K-26056 (2.5 mg) |
| (minutes) | directly after production | after 6 weeks at 40° C./75% RH |
| 15 | 97 | 98 |
| 30 | 97 | 97 |
| 45 | 97 | 97 |
| 60 | 97 | 97 |

EXAMPLE 3B
Dissolution of 5 mg High Purity Torsemide modification II Tablets

| | Torsemide dissolved (%) | |
|---|---|---|
| Time | K-26057 (5 mg) | K-26057 (5 mg) |
| (minutes) | directly after production | after 6 weeks at 40° C./75% RH |
| 15 | 98 | 95 |
| 30 | 98 | 96 |
| 45 | 97 | 95 |
| 60 | 99 | 95 |

EXAMPLE 3C
Dissolution of 100 mg High Purity Torsemide modification II Tablets

| | Torsemide dissolved (%) | |
|---|---|---|
| Time | K-26058 (100 mg) | K-26058 (100 mg) |
| (minutes) | directly after production | after 6 weeks at 40° C./75% RH |
| 15 | 87 | 81 |
| 30 | 92 | 84 |
| 45 | 93 | 89 |
| 60 | 93 | 89 |

### Example 4

### Dissolution Results

The dissolution method used was the U.S.P. Paddle Method, at 50 RPM with purified water at 37° C. For the dissolution test, 6 tablets were tested in 900 mL of purified water according to the Paddle Method of the U.S.P. Example 4B shows the dissolution rates of one representative tablet lot directly after production and after 3 months of storage at 40° C. at a relative humidity (RH) of 75%. The dissolution rates of the high purity torsemide modification II tablet Batch No. K-26683 were identical under both conditions. There was no substantial change in the dissolution rates of any of the present pharmaceutical formulations containing high purity torsemide modification II following 3 months at the above storage conditions.

EXAMPLE 4A
High purity Torsemide Tablets (100 mg)

| Composition (Batch No. K-26683) | grams per 3,750 tablets |
|---|---|
| High purity Torsemide modification II | 375.0 |
| Lactose Anhydrous NF | 382.5 |
| Crospovidone NF | 180.0 |
| Povidone USP (PVP K-30) | 22.5 |
| Microcrystalline Cellulose NF (Avicel PH 112) | 525 |
| Alcohol USP | 620.00 |
| Magnesium Stearate NF | 15.0 |

EXAMPLE 4B
Dissolution of 100 mg High purity Torsemide modification II Tablets

| | Torsemide dissolved (%) | |
|---|---|---|
| Time | K-26683 (100 mg) | K-26683 (100 mg) |
| (minutes) | directly after production | after 3 months at 40° C./75% RH |
| 15 | 88 | 88 |
| 30 | 97 | 92 |
| 45 | 98 | 97 |
| 60 | 99 | 98 |

### Example 5

### Preparation of High Purity Torsemide Modification II

First step: Preparation of Torsemide Modification I

A 100 mL three necked flask, equipped with thermometer and a mechanical stirrer was charged with a mixture of acetonitrile:water (5:1, 26 mL), and torsemide (modification II containing less than 20% of modification I, 5 grams) and stirred at 60° C. for 30 minutes. The mixture was then filtered hot and washed using the same solvent mixture (2×6.5 mL). The wet solid dried under high vacuum (3 mm Hg) at 50° C. for 6 hours to yield 4.7 grams of torsemide modification I in which no torsemide modification II was detectable by IR or x-ray powder diffraction methods.

Second Step:

A 5 L three necked flask equipped with a mechanical stirrer and a pH meter electrode, was charged with water (3,000 L) and torsemide modification I (177 grams). The pH of the solution was adjusted to 10.2±0.2 with 20% NaOH (approximately 53 mL). The solution is then filtered and the pH of the solution was adjusted with approximately 66 mL of a 1:1 acetic acid:water solution to a pH of 6.25±0.2. The white precipitate was filtered and washed with water (2×50 mL) and dried in a high vacuum oven at 50° C. for 6 hours. High Purity Torsemide modification II was isolated in 93.2% yield, 165 grams.

Although certain presently preferred embodiments of the invention have been described herein, it will be apparent to those skilled in the art to which the invention pertains that variations and modifications of the described embodiment may be made without departing from the spirit and scope of the invention. Accordingly, it is intended that the invention be limited only to the extent required by the appended claims and the applicable rules of law.

What is claimed is:

1. A process for making high purity torsemide modification II comprising the steps of:
(a) adding torsemide modification I to a solvent mixture comprising acetonitrile and water;
(b) isolating torsemide modification I;
(c) suspending the torsemide modification I of step (b) in water to form a solution;
(d) adjusting the solution of step (c) to a pH of about 10±0.2;
(e) filtering the solution of step (d);
(f) adjusting the solution of step (e) to a pH of 6.25±0.2; and
(g) isolating high purity torsemide modification II.

2. A stable pharmaceutical formulation comprising an effective amount of torsemide modification II and a pharmaceutically acceptable excipients wherein the excipients have a low moisture content.

3. The stable pharmaceutical formulation of claim 2 further comprising the excipients having a low moisture content selected from the group consisting of lactose anhydrous, crospovidone, povidone, microcrystalline cellulose, and magnesium stearate.

4. The stable pharmaceutical formulation of claim 2 comprising torsemide modification II in an amount of about 2.5 mg to about 200 mg per tablet.

5. The stable pharmaceutical formulation of claim 4 comprises torsemide modification II in an amount of about 2.5 mg, about 5 mg, about 10 mg, about 20 mg or about 100 mg per tablet.

6. A stable pharmaceutical formulation comprising an effective amount of torsemide modification II wherein the torsemide modification II does not substantially rearrange into another form of torsemide over time upon storage.

7. The stable pharmaceutical formulation of claim 6 wherein the formulation is stored under stress conditions.

8. The stable pharmaceutical formulation of claim 7 wherein the formulation is stored at about 40° C. and about 75% relative humidity.

9. The stable pharmaceutical formulation of claim 6 wherein the torsemide modification II does not substantially rearrange into torsemide modification I over time upon storage under stress conditions.

10. The stable pharmaceutical formulation of claim 9 wherein not more than 5% of the torsemide modification II rearranges into torsemide modification I.

11. The stable pharmaceutical formulation of claim 6 wherein the torsemide modification II is selected from the group consisting of high purity torsemide modification II and torsemide modification II containing trace amounts of torsemide modification I.

12. The stable pharmaceutical formulation of claim 11 wherein the torsemide modification II comprises about 0.5 to about 2% (w/w) of torsemide modification I.

13. The stable pharmaceutical formulation of claim 6 wherein the torsemide modification II has a particle size distribution such that 100% is below 200%.

14. The stable pharmaceutical formulation of claim 13 wherein the particle size distribution is such that 100% is below 100μ.

15. The stable pharmaceutical formulation of claim 14 wherein the particle size distribution is such that 100% is below 50μ.

16. High purity torsemide modification II.

17. The high purity torsemide modification II of claim 16 which is a stable polymorphic form of torsemiide.

18. The high purity torsemide modification II of claim 17 which does not substantially rearrange over times.

19. The high purity torsemide modification II of claim 18 further characterized by being stable during storage under stress conditions for at least 3 months.

20. The high purity torsemide modification II of claim 18 which is in the form of fine crystal.

21. The high purity torsemide modification II of claim 18 wherein the high purity torsemide modification II does not substantially rearrange over time into torsemide modification I.

22. The high purity torsemide modification II of claim 21 wherein not more than 10% of the high purity torsemide modification II rearranges over time into torsemide modification I.

23. The high purity torsemide modification II of claim 17 which is further characterized by having a particle size distribution such that 100% is below 200μ.

24. The high purity torsemide modification II of claim 23 which is further characterized by having a particle size distribution such that 100% is below 100μ.

25. The high purity torsemide modification II of claim 24 which is further characterized by having a particle size distribution such that 100% is below 50μ.

26. High purity torsemide modification II produced according to the process of claim 1.

27. The high purity torsemide modification II of claim 26 which is a stable polymorphic form of torsemide.

28. The high purity torsemide modification II of claim 27 which does not substantially rearrange over times.

29. The high purity torsemide modification II of claim 28 further characterized by being stable during storage under stress conditions for at least 3 months.

30. The high purity torsemide modification II of claim 28 which is in the form of fine crystal.

31. The high purity torsemide modification II of claim 28 wherein the high purity torsemide modification II does not substantially rearrange over time into torsemide modification I.

32. The high purity torsemide modification II of claim 31 wherein not more than 10% of the high purity torsemide modification II rearranges over time into torsemide modification I.

33. The high purity torsemide modification II of claim 32 which is further characterized by having a particle size distribution such that 100% is below 200μ.

34. The high purity torsemide modification II of claim 33 which is further characterized by having a particle size distribution such that 100% is below 100μ.

35. The high purity torsemide modification II of claim 34 which is further characterized by having a particle size distribution such that 100% is below 50μ.
